(19) United States
(12) Patent Application Publication
(10) Pub. No.: US 2025/0251371 A1
(43) Pub. Date: Aug. 7, 2025

(54) Method for Identifying Calculus Bovis and Substitute Thereof, and Use Thereof

(71) Applicant: Anhui University of Chinese Medicine, Hefei City (CN)

(72) Inventors: Can PENG, Hefei City (CN); Chijing ZUO, Hefei City (CN); Gege YANG, Hefei City (CN); Yulong ZHU, Hefei City (CN); Jiazhi YAN, Hefei City (CN); Jing ZHANG, Hefei City (CN); Shiting LI, Hefei City (CN); Zihao HUANG, Hefei City (CN); Xiangxiang YU, Hefei City (CN); Yunjing ZHANG, Hefei City (CN)

(21) Appl. No.: 19/033,751

(22) Filed: Jan. 22, 2025

(30) Foreign Application Priority Data
Feb. 2, 2024 (CN) 202410149627.7

(51) Int. Cl. G01N 30/72; G01N 30/06; G01N 33/50

(52) U.S. Cl. CPC G01N 30/7233; G01N 30/06; G01N 30/8679; G01N 33/50; G01N 33/92

(57) ABSTRACT

Provided is a method for identifying Calculus Bovis and a substitute thereof, and use thereof. The method includes the following steps: subjecting a cholic acid (CA) component of the Calculus Bovis and the substitute thereof to qualitative and quantitative analysis by ultra-performance liquid chromatography-mixed quadrupole orbitrap mass spectrometry (UPLC-Q-orbitrap-MS), and then establishing a chromatogram of the CA component of the Calculus Bovis and the substitute thereof; screening a significantly different CA component in the Calculus Bovis and the substitute thereof by multivariate statistical analysis (MVSA); and establishing a discriminant function equation for the Calculus Bovis and the substitute thereof to identify the Calculus Bovis and the substitute thereof. The method provides a novel and simple evaluation standard for a quality control of Chinese patent medicines, and a reference for the construction of a quality control system of Chinese medicine compound prescriptions.

### CROSS REFERENCE TO RELATED APPLICATION

This patent application claims the benefit and priority of Chinese Patent Application No. 202410149627.7 filed with the China National Intellectual Property Administration on Feb. 2, 2024, the disclosure of which is incorporated by reference herein in its entirety as part of the present application.

### TECHNICAL FIELD

The present disclosure belongs to the technical field of traditional Chinese medicine identification, and particularly relates to a method for identifying Calculus Bovis and a substitute thereof, and use thereof.

### BACKGROUND

Calculus Bovis, a dried gallstone obtained from the gallbladder or bile duct of cattle, is a traditional and precious Chinese medicinal material, and has substitutes including ox bile powder, Calculus Bovis Artifactus, and Calculus Bovis Sativus. The price of Calculus Bovis and substitute thereof varies greatly, and are difficult to be differentiated due to the similarity of cholic acid (CA) components, complex structure, and a large number of isomers. Moreover, numerous traditional Chinese medicine formulations incorporate Calculus Bovis and substitute thereof as key components. Some merchants indicate the type of Calculus Bovis used when selling, but some unscrupulous merchants can deliberately mix, adulterate, and falsify in order to save costs or cut corners, which seriously affect the stability of the Calculus Bovis medicinal material market. Therefore, it is necessary to conduct corresponding research work to establish an accurate and direct identification method for Calculus Bovis and substitutes thereof and related patent medicines.

In recent years, numerous domestic and international studies have reported methods for identifying Calculus Bovis and substitute medicinal materials thereof, mainly including chromogenic method, infrared (near infrared) spectroscopy, thin-layer chromatography (TLC) scanning, ultraviolet (UV) spectrophotometry, differential thermal analysis (DTA), powder X-ray diffraction, and high-performance liquid chromatography (HPLC). In addition, some scholars have also developed novel technologies, such as determining CA components of the Calculus Bovis Artifactus by micellar electrokinetic capillary electrophoresis. Due to the complexity of the ingredients in Calculus Bovis medicinal materials and their similar properties, the mentioned methods exhibit certain limitations.

The chromogenic method is not specific and cannot reflect all the information of a sample since its analysis results are restricted by the chromatographic analysis conditions and extraction method. Therefore, it is difficult to identify the Calculus Bovis and substitutes fully and accurately. Many types of the infrared (near infrared) spectroscopy are mid-infrared frequency doublings, which have large interference between spectra, making analysis difficult. The TLC scanning is relatively suitable for the determination of CA components in multi-component Chinese patent medicines, rather than a single medicinal material, and is thus difficult to identify the Calculus Bovis and substitutes thereof. Although the UV spectrophotometry is easy to operate, the experimental conditions such as water bath time and temperature have a certain impact on the experimental results, and its reproducibility needs to be further investigated. Although the DTA can find significant differences in the thermal spectra between different types of Calculus Bovis samples, there are strict requirements on external conditions, intuitiveness lack, and difficulty in identifying patent medicines. The powder X-ray diffraction can distinguish the Calculus Bovis from Calculus Bovis Artifactus according to spectra and diffraction peaks, but has slightly complicated operations, difficulty in making absolute analysis, low sensitivity to light elements, and easily affected by mutual element interference and superposition peaks. The HPLC, which can be connected to various types of detectors, has desirable reproducibility and high accuracy, and can better meet the quality control requirements of Calculus Bovis and substitutes thereof. However, the operation of this method is relatively complicated, and there are many influencing factors, making the results of HPLC uncertain. The micellar electrokinetic capillary electrophoresis is relatively complex to operate with high requirements on the external environment.

### SUMMARY

In order to solve the above technical problems, an objective of the present disclosure is to provide a method for identifying Calculus Bovis and a substitute thereof. The method can intuitively, quickly and accurately identify the Calculus Bovis and the substitute thereof by combining ultra-performance liquid chromatography-mixed quadrupole orbitrap mass spectrometry (UPLC-Q-orbitrap-MS) with multivariate statistical analysis (MVSA).

To achieve the above objective, the present disclosure adopts the following technical solutions:

The present disclosure provides a method for identifying Calculus Bovis and a substitute thereof, including the following steps:
(1) subjecting a CA component of the Calculus Bovis and the substitute thereof to qualitative and quantitative analysis by UPLC-Q-orbitrap-MS, and then establishing a chromatogram of the CA component of the Calculus Bovis and the substitute thereof;
(2) screening a significantly different CA component in the Calculus Bovis and the substitute thereof by MVSA; and
(3) establishing a discriminant function equation for the Calculus Bovis and the substitute thereof to identify the Calculus Bovis and the substitute thereof.

In some embodiments, chromatographic conditions in step (1) include: a C18 chromatographic column; a mobile phase A of 5 mmol·L⁻¹ ammonium acetate solution, and a mobile phase B of methanol solution; a gradient elution program including: 60% B from 0 min to 2 min; 60% to 64% B from 2 min to 18 min; 64% to 95% B from 18 min to 19 min; 95% B from 19 min to 21 min; 95% to 60% B from 21 min to 22 min; and 60% B from 22 min to 24 min.

In some embodiments, mass spectrometry conditions in step (1) include: an electrospray ionization (ESI) source, operation with a negative ion in a parallel reaction monitoring (PRM) mode, and a spray voltage of −3.8 kV; an ion source temperature of 380° C.; a sheath gas flow rate of 30 μL·min⁻¹; an auxiliary gas flow rate of 5 μL·min⁻¹; and an auxiliary gas N₂ heater temperature of 300° C.

In some embodiments, the MVSA in step (2) is one or more selected from the group consisting of hierarchical cluster analysis (HCA), principal component analysis (PCA), and orthogonal partial least squares-discriminant analysis (OPLS-DA).

In some embodiments, the significantly different CA component in step (2) is selected from the group consisting of CA, taurodeoxycholic acid (TDCA), taurolithocholic acid (TLCA), glycochenodeoxycholic acid (GCDCA), hyodeoxycholic acid (HDCA), and deoxycholic acid (DCA).

In some embodiments, the discriminant function equation of the Calculus Bovis and the substitute thereof in step (3) are:

$$Y 1 = - 0.0 \; 34 S 1 + 0.3 \; 45 S 2 - 3.071 S 3 + 0.1 \; 21 S 4 + 0.5 \; 15 S 5 + 0.0 \; 71 S 6 - 91.575 ;$$

$$Y 2 = 0.2 \; 53 S 1 + 2.1 \; 42 S 2 + 4.9 \; 59 S 3 + 3.1 \; 61 S 4 - 0.446 S 5 - 0.207 S 6 - 401.886 ;$$

$$Y 3 = 0. 0 \; 15 S 1 + 0.0 \; 37 S 2 - 0.112 S 3 + 0.1 \; 45 S 4 - 0.023 S 5 + 0.0 \; 23 S 6 - 1.997 ;$$

$$Y 4 = 0. 4 \; 72 S 1 - 0.783 S 2 + 5.0 \; 61 S 3 + 2.3 \; 61 S 4 - 0.619 S 5 + 0.3 \; 79 S 6 - 204. 8 \; 36 ;$$

Y1 represents Calculus Bovis Artifactus, Y2 represents an ox bile powder, Y3 represents the Calculus Bovis, and Y4 represents Calculus Bovis Sativus; and
S1 represents CA, S2 represents TDCA, S3 represents TLCA, S4 represents GCDCA, S5 represents HDCA, and S6 represents DCA.

The present disclosure provides use of the method for identifying Calculus Bovis and a substitute thereof in quality control of a medicinal material and a Chinese patent medicine thereof.

Compared with the prior art, the present disclosure has the following excellent effects:

In the present disclosure, the method adopts UPLC-Q-orbitrap-MS combined with MVSA to screen out a significantly different CA component in the Calculus Bovis and the substitute thereof, establishes a discriminant model equation, traces a source of the medicinal material, and systematically reveals differences between the Calculus Bovis and the substitute thereof. The method can not only intuitively, quickly, and accurately identify the medicinal materials of the Calculus Bovis and the substitute thereof, but also provide an experimental basis for further studying the quality of related patent medicines containing the Calculus Bovis and the substitute thereof. Therefore, a novel and simple evaluation standard is proposed for the quality control of Chinese patent medicines, and a reference is also provided for the construction of a quality control system of Chinese medicine compound prescriptions.

### BRIEF DESCRIPTION OF THE DRAWINGS

FIG. 1 shows the typical Parallel Reaction Monitoring (PRM) extracted ion chromatogram of a standard solution;

FIG. 2 shows the typical PRM extracted ion chromatogram of Calculus Bovis Artifactus;

FIG. 3 shows the typical PRM extracted ion chromatogram of an ox bile powder;

FIG. 4 shows the typical PRM extracted ion chromatogram of Calculus Bovis;

FIG. 5 shows the typical PRM extracted ion chromatogram of Calculus Bovis Sativus;

FIG. 6 shows the HCA graphs of 32 batches of Calculus Bovis samples and substitutes thereof;

FIG. 7 shows the PCA score plots of 32 batches of Calculus Bovis samples and substitutes thereof;

FIG. 8 shows the PCA loading diagram of 17 CA components of Calculus Bovis samples and substitutes thereof;

FIG. 9 shows the S-plot loading diagram of OPLS-DA on 15 CA components of the Calculus Bovis Artifactus and ox bile powder;

FIG. 10 shows the S-plot loading diagram of OPLS-DA on 15 CA components of the Calculus Bovis Artifactus and Calculus Bovis;

FIG. 11 shows the S-plot loading diagram of OPLS-DA on 14 CA components of the Calculus Bovis Artifactus and Calculus Bovis Sativus;

FIG. 12 shows the S-plot loading diagram of OPLS-DA on 16 CA components of the ox bile powder and Calculus Bovis;

FIG. 13 shows the S-plot loading diagram of OPLS-DA on 15 CA components of the ox bile powder and Calculus Bovis Sativus; and

FIG. 14 shows the S-plot loading diagram of OPLS-DA on 15 CA components of the Calculus Bovis and Calculus Bovis Sativus.

### DETAILED DESCRIPTION OF THE EMBODIMENTS

The present disclosure is further described below by detailed description. The following examples are detailed descriptions of the present disclosure, but the embodiments of the present disclosure are not limited to the following examples.

### Example 1: Identification Method of Calculus Bovis and Substitutes Thereof

### 1. Establishment of a Method for Detecting CA Components Based on UPLC-Q-Orbitrap-MS

### 1.1 Reagents

Standards of chenodeoxycholic acid (CDCA, batch number 110806-201507, purity≥91.3%), HDCA (batch number 100087-201411, purity≥99.7%), ursodeoxycholic acid (UDCA, batch number 110755-201704, purity≥99.4%), deoxycholic acid (DCA, batch number AZ7UN-AH, purity≥98%), tauroursodeoxycholic acid (TUDCA, batch number 110816-201509, purity≥93.4%), taurochenodeoxycholic acid (TCDCA, batch number 110846-201007, purity≥97%), taurohyodeoxycholic acid (THDCA, batch number 111943-201802, purity≥96.8%), TDCA (batch number CHB201115, purity≥97%), GCDCA (batch number ZZS18072612, purity≥97%), glycoursodeoxycholic acid (GUDCA, batch number CHB201112, purity≥95%), glycohyodeoxycholic acid (GHDCA, batch number CHB210121, purity≥98%), glycodeoxycholic acid (GDCA, batch number ZZS19017125, purity≥97%), glycocholic acid (GCA, batch number ZZS18072611, purity≥97%), taurocholic acid (TCA, batch number CHB180309, purity≥98%), CA (batch number 100078-201415, purity≥98.9%), lithocholic acid (LCA, batch number FCB055902, purity≥99%), taurolithocholic acid (TLCA, batch number CHB201116, purity≥97%), and deuterated CA (batch number E-816204-ND1, purity≥99%) were purchased from Chengdu Chroma-Biotechnology Co., Ltd.; methanol (mass spectrometry-grade) was purchased from Sigma-Aldrich (Shanghai) Trading Co., Ltd., batch number 11112807036; ammonium acetate was purchased from Fisher Chemical Reagent Company, USA, batch number 183355; microporous filter membrane (0.22 um) was provided by Millipore (Billerica, MA, USA); water was ultrapure water prepared by a laboratory water purification system (PALL, USA).

### 1.2 Methods and Results

### 1.2.1 Preparation of Standard Stock Solutions

2 mg of cach of the standards of CDCA, HDCA, UDCA, DCA, TUDCA, TCDCA, THDCA, TDCA, GCDCA, GUDCA, GHDCA, GDCA, GCA, TCA, CA, LCA, and TLCA werc accurately weighed and added in 2 mL brown volumetric flasks, added with mass spectrometry-grade methanol to dissolve and dilute to the scale, and ultrasonicated for 30 min to prepare standard stock solutions containing 1 mg·mL⁻¹ of the standards, respectively. 1 mg of deuterated CA was added in a 1 mL brown volumetric flask, diluted to scale, and ultrasonicated for 30 min to prepare a 1 mg·mL⁻¹ internal standard solution for later use. All solutions were operated in the dark and stored at 4° C. until analysis.

### 1.2.2 Preparation of Mixed Standard Solution

The standard stock solutions prepared under “1.2.1 Preparation of standard stock solutions” were taken and mixed to prepare a mixed standard solution containing 100 ng·mL⁻¹ of each standard (including internal standard solution), mixed under vortex, and centrifuged for later use. All solutions were operated in the dark and stored at 4° C. until analysis.

### 1.2.3 Chromatographic Conditions

A Hypersil Gold C18 column (100×2.1 mm, 1.9 μm) was used; the mobile phase A was 5 mmol·L⁻¹ ammonium acetate solution, B was methanol solution, and the gradient elution program included: 60% B from 0 min to 2 min; 60% to 64% B from 2 min to 18 min; 64% to 95% B from 18 min to 19 min; 95% B from 19 min to 21 min; 95% to 60% B from 21 min to 22 min; and 60% B from 22 min to 24 min; flow rate 0.2 mL·min⁻¹; column temperature 40° C.; injection volume 2 μL.

### 1.2.4 MS Conditions

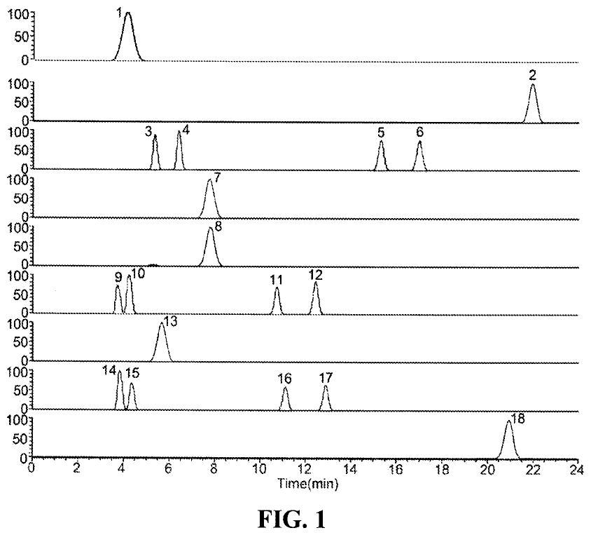

The mass spectrometer detector was equipped with an ESI source, which could operate in positive and negative ion modes. Xcalibur 2.1 software (Thermo Fisher Scientific, Waltham, MA, USA) was used and the mass spectrometer was operated with a negative ion in a PRM mode. In order to achieve maximum sensitivity, the mass spectrometry conditions were optimized: a spray voltage of-3.8 kV; an ion source temperature of 380° C.; a sheath gas flow rate of 30 μL·min⁻¹; an auxiliary gas flow rate of 5 μL·min⁻¹; and an auxiliary gas N2 heater temperature of 300° C. The robustness of the method was tested by quality control on every 10 samples. The PRM extracted ion chromatogram of the of the standard solution was shown in FIG. 1, and the compound ion pairs, optimized collision energy (CE) and other information were shown in Table 1.

TABLE 1
MS-MS detection parameters for each analyte

| | Relative | | [M − | | |
|---|---|---|---|---|---|
| | molecular | | H]⁻ | PRM | |
| Component | mass | t_{R}(min) | (m/z) | conversion | CE(V) |
| CDCA | C₂₄H₄₀O₄ | 15.32 | 391.3 | 391.3→391.3 | 30 |
| HDCA | C₂₄H₄₀O₄ | 6.38 | 391.3 | 391.3→391.3 | 30 |
| UDCA | C₂₄H₄₀O₄ | 5.32 | 391.3 | 391.3→391.3 | 30 |
| DCA | C₂₄H₄₀O₄ | 16.99 | 391.3 | 391.3→391.3 | 30 |
| TUDCA | C₂₆H₄₅NO₆S | 3.72 | 498.3 | 498.3→80 | 30 |
| TCDCA | C₂₆H₄₅NO₆S | 10.75 | 498.3 | 498.3→80 | 30 |
| THDCA | C₂₆H₄₅NO₆S | 4.21 | 498.3 | 498.3→80 | 30 |
| TDCA | C₂₆H₄₅NO₆S | 12.43 | 498.3 | 498.3→80 | 30 |
| GCDCA | C₂₆H₄₃NO₅ | 11.1 | 448.3 | 448.3→74.1 | 20 |
| GUDCA | C₂₆H₄₃NO₅ | 3.78 | 448.3 | 448.3→74.1 | 20 |
| GHDCA | C₂₆H₄₃NO₅ | 4.34 | 448.3 | 448.3→74.1 | 20 |
| GDCA | C₂₆H₄₃NO₅ | 12.89 | 448.3 | 448.3→74.1 | 20 |
| GCA | C₂₆H₄₃NO₆ | 3.68 | 464.3 | 464.3→74.1 | 20 |
| TCA | C₂₆H₄₅NO₇S | 5.66 | 514.3 | 514.3→80 | 30 |
| CA | C₂₄H₄₀O₅ | 7.77 | 407.3 | 407.3→407.3 | 40 |
| LCA | C₂₄H₄₀O₃ | 21.92 | 375.3 | 375.3→375.3 | 30 |
| TLCA | C₂₆H₄₅NO₅S | 20.95 | 482.3 | 482.3→80 | 30 |

### 2. Qualitative and Quantitative Analysis of CA Components in Calculus Bovis and Substitutes Thereof

### 2.1 Sample Information

A total of 32 batches of experimental samples were collected, and the specific sample information was recorded in Table 2. 8 batches of Calculus Bovis Artifactus and 8 batches of ox bile powder were provided by Anhui Bozhou Huatuo National Pharmaceutical Co., Ltd. and Wuhan Baicaoyuan Biochemical Pharmaceutical Co., Ltd.; 8 batches of Calculus Bovis were provided by Sichuan Aba Prefecture Pharmaceutical and Medicinal Materials Corporation and Guangdong Medicinal Materials Company; 8 batches of Calculus Bovis Sativus were provided by Wuhan Jianmin Dapeng Pharmaceutical Co., Ltd.

TABLE 2
Sample information

| Number | Sample | Batch No. | Source |
|---|---|---|---|
| A-1 | Calculus Bovis | 20180901 | Anhui Bozhou Huatuo National |
| | Artifactus | | Pharmaceutical Co., Ltd. |
| A-2 | Calculus Bovis | 20180503 | Anhui Bozhou Huatuo National |
| | Artifactus | | Pharmaceutical Co., Ltd. |
| A-3 | Calculus Bovis | 20180903 | Anhui Bozhou Huatuo National |
| | Artifactus | | Pharmaceutical Co., Ltd. |
| A-4 | Calculus Bovis | 20180502 | Anhui Bozhou Huatuo National |
| | Artifactus | | Pharmaceutical Co., Ltd. |
| A-5 | Calculus Bovis | 210302 | Wuhan Baicaoyuan Biochemical |
| | Artifactus | | Pharmaceutical Co., Ltd. |
| A-6 | Calculus Bovis | 210306 | Wuhan Baicaoyuan Biochemical |
| | Artifactus | | Pharmaceutical Co., Ltd. |
| A-7 | Calculus Bovis | 210305 | Wuhan Baicaoyuan Biochemical |
| | Artifactus | | Pharmaceutical Co., Ltd. |
| A-8 | Calculus Bovis | 210301 | Wuhan Baicaoyuan Biochemical |
| | Artifactus | | Pharmaceutical Co., Ltd. |
| B-1 | Ox bile powder | 20180502 | Anhui Bozhou Huatuo National |
| | | | Pharmaceutical Co., Ltd. |
| B-2 | Ox bile powder | 20180503 | Anhui Bozhou Huatuo National |
| | | | Pharmaceutical Co., Ltd. |
| B-3 | Ox bile powder | 20180901 | Anhui Bozhou Huatuo National |
| | | | Pharmaceutical Co., Ltd. |
| B-4 | Ox bile powder | 20180903 | Anhui Bozhou Huatuo National |
| | | | Pharmaceutical Co., Ltd. |
| B-5 | Ox bile powder | B201201-1 | Wuhan Baicaoyuan Biochemical |
| | | | Pharmaceutical Co., Ltd. |
| B-6 | Ox bile powder | B201201-2 | Wuhan Baicaoyuan Biochemical |
| | | | Pharmaceutical Co., Ltd. |
| B-7 | Ox bile powder | B201202-1 | Wuhan Baicaoyuan Biochemical |
| | | | Pharmaceutical Co., Ltd. |
| B-8 | Ox bile powder | B201202-2 | Wuhan Baicaoyuan Biochemical |
| | | | Pharmaceutical Co., Ltd. |
| C-1 | Calculus Bovis | 2N3 | Sichuan Aba Prefecture |
| | | | Pharmaceutical and Medicinal |
| | | | Materials Corporation |
| C-2 | Calculus Bovis | 2P4 | Sichuan Aba Prefecture |
| | | | Pharmaceutical and Medicinal |
| | | | Materials Corporation |
| C-3 | Calculus Bovis | 2Q2 | Sichuan Aba Prefecture |
| | | | Pharmaceutical and Medicinal |
| | | | Materials Corporation |
| C-4 | Calculus Bovis | 2N5 | Sichuan Aba Prefecture |
| | | | Pharmaceutical and Medicinal |
| | | | Materials Corporation |
| C-5 | Calculus Bovis | 201105-1 | Guangdong Medicinal |
| | | | Materials Company |
| C-6 | Calculus Bovis | 201105-2 | Guangdong Medicinal |
| | | | Materials Company |
| C-7 | Calculus Bovis | 201105-3 | Guangdong Medicinal |
| | | | Materials Company |
| C-8 | Calculus Bovis | 201105-4 | Guangdong Medicinal |
| | | | Materials Company |
| D-1 | Calculus Bovis | 200501 | Wuhan Jianmin Dapeng |
| | Sativus | | Pharmaceutical Co., Ltd. |
| D-2 | Calculus Bovis | 201103 | Wuhan Jianmin Dapeng |
| | Sativus | | Pharmaceutical Co., Ltd. |
| D-3 | Calculus Bovis | 201102 | Wuhan Jianmin Dapeng |
| | Sativus | | Pharmaceutical Co., Ltd. |
| D-4 | Calculus Bovis | 191103 | Wuhan Jianmin Dapeng |
| | Sativus | | Pharmaceutical Co., Ltd. |
| D-5 | Calculus Bovis | 191104 | Wuhan Jianmin Dapeng |
| | Sativus | | Pharmaceutical Co., Ltd. |
| D-6 | Calculus Bovis | 190406 | Wuhan Jianmin Dapeng |
| | Sativus | | Pharmaceutical Co., Ltd. |
| D-7 | Calculus Bovis | 190903 | Wuhan Jianmin Dapeng |
| | Sativus | | Pharmaceutical Co., Ltd. |
| D-8 | Calculus Bovis | 191201 | Wuhan Jianmin Dapeng |
| | Sativus | | Pharmaceutical Co., Ltd. |

### 2.2 Methods and Results

### 2.2.1 Preparation of Standard Stock Solutions

The preparation of standard stock solution was the same as that in “1.2.1 Preparation of standard stock solutions”. In addition, the 1 mg·mL⁻¹ deuterated CA internal standard solution prepared under “1.2.1 Preparation of standard stock solutions” needed to be diluted step by step to a 1 μg·mL⁻¹ stock solution for use. All solutions were operated in the dark and stored at 4° C. until analysis.

### 2.2.2 Preparation of Standard Curve of Mixed Standard Solution

1 mg·mL⁻¹ CA standard stock solution was diluted 2.5 times to 400 μg mL⁻¹ for use; 100 μL of each of 1 mg mL⁻¹ HDCA, LCA, GCA, TCA, and DCA was taken to prepare a solution containing 200 μg·mL⁻¹ of each standard for use; 1 mg·mL⁻¹ CDCA, GDCA, GCDCA, GHDCA, TCDCA, TDCA, and UDCA were diluted 2.5 times to 400 μg·mL^{-1,} and then 100 μL of each was added to 300 μL of mass spectrometry-grade methanol to dilute to a 40 μg mL⁻¹ solution for use; 1 mg·mL⁻¹ GUDCA, TUDCA, THDCA, and TLCA were diluted to 100 μg mL⁻¹ respectively, and then 100 μL of each above was added with 600 μL of mass spectrometry-grade methanol to dilute to a 10 μg mL⁻¹ solution for use.

The above-mentioned stock solutions were swirled evenly and mixed well. 200 μL of each of the four stock solutions was taken and swirled evenly and mixed to prepare a stock solution, containing 100 μg mL⁻¹ of CA, 50 μg mL⁻¹ of HDCA, LCA, GCA, TCA, and DCA, 10 μg mL⁻¹ of CDCA, GDCA, GCDCA, GHDCA, TCDCA, TDCA, and UDCA, and 2.5 μg mL⁻¹ of GUDCA, TUDCA, THDCA, and TLCA.

The stock solution containing each standard was diluted stepwise, and finally 8 concentration gradients were determined, including CA (2 ng·mL⁻¹, 10 ng·mL⁻¹, 50 ng·mL⁻¹, 200 ng·mL⁻¹, 800 ng·mL⁻¹, 4 μg mL⁻¹, 20 μg·mL⁻¹, 100 μg·mL⁻¹); HDCA, LCA, GCA, TCA, and DCA (1 ng·mL⁻¹, 5 ng·mL⁻¹, 25 ng·mL⁻¹, 100 ng·mL⁻¹, 400 ng·mL⁻¹, 2 μg·mL⁻¹, 10 μg·mL⁻¹, 50 μg·mL⁻¹); CDCA, GDCA, GCDCA, GHDCA, TCDCA, TDCA, and UDCA (0.2 ng·mL⁻¹, 1 ng·mL⁻¹, 5 ng·mL⁻¹, 20 ng·mL⁻¹, 80 ng·mL⁻¹, 400 ng·mL⁻¹, 2 μg·mL⁻¹, 10 μg·mL⁻¹); GUDCA, TUDCA, THDCA, and TLCA (0.05 ng·mL⁻¹, 0.25 ng·mL⁻¹, 1.25 ng·mL⁻¹, 5 ng·mL⁻¹, 20 ng·mL⁻¹, 100 ng·mL⁻¹, 500 ng·mL⁻¹, 2.5 μg·mL⁻¹).

8 gradient solutions was mixed with the 1 μg·mL⁻¹ deuterated CA internal standard solution prepared in “2.2.1 Preparation of standard stock solutions”, that is, 100 μL of each of the 8 gradient solutions, added with 100 μL of the internal standard solution and 800 μL of mass spectrometry-grade methanol, mixed well under vortex to prepare a standard curve, including CA (0.2 ng·mL⁻¹, 1 ng·mL⁻¹, 5 ng·mL⁻¹, 20 ng·mL⁻¹, 80 ng·mL⁻¹, 400 ng·mL⁻¹, 2 μg·mL⁻¹, 10 μg·mL⁻¹); HDCA, LCA, GCA, TCA, and DCA (0.1 ng·mL⁻¹, 0.5 ng·mL⁻¹, 2.5 ng·mL⁻¹, 10 ng·mL⁻¹, 40 ng·mL⁻¹, 200 ng·mL⁻¹, 1 μg·mL⁻¹, 5 μg·mL⁻¹); CDCA, GDCA, GCDCA, GHDCA, TCDCA, TDCA, and UDCA (0.02 ng·mL⁻¹, 0.1 ng·mL⁻¹, 0.5 ng·mL⁻¹, 2 ng·mL⁻¹, 8 ng·mL⁻¹, 40 ng·mL⁻¹, 200 ng·mL⁻¹, 1 μg·mL⁻¹); GUDCA, TUDCA, THDCA, and TLCA (0.005 ng·mL^{-1, 0.025} ng·mL⁻¹, 0.125 ng·mL⁻¹, 0.5 ng·mL⁻¹, 2 ng·mL⁻¹, 10 ng·mL⁻¹, 50 ng·mL⁻1, 250 ng·mL⁻¹); in which the internal standard solution was 100ng·mL⁻¹ respectively. All solutions were operated in the dark and stored at 4° C. until analysis.

### 2.2.3 Preparation of Test Solutions

5 mg of sample powder was accurately weighed, and placed in 10 mL brown volumetric flasks. The prepared solvent (DMSO-CHCL₃—CH₃OH, 2:2:1, v/v, prepared and used immediately) was added to the scale, and the sealing film was tightly plugged. Ultrasonication (power of 250 W, frequency of 40 Hz) was conducted at 4° C. for 30 min in ice bath, and a resulting mixture was allowed to stand for 30 min, and then centrifuged (1,250 rpm, 10 min). The supernatant was taken 10) and placed in a 10 mL EP tube. 100 μL of the supernatant was taken, diluted ten-fold and placed in a 2 mL EP tube to obtain a diluted sample. Then 100 μL of the diluted sample was taken and placed in a 2 mL EP tube, added with 100 μL of the internal standard solution prepared in “2.2.1 Preparation of standard stock solutions”, and then mixed with 800 μL of mass spectrometry-grade methanol to obtain a sample solution of 5 μg·mL⁻¹. The sample solution was centrifuged (1,250 rpm, 10 min), a resulting supernatant was taken, and filtered with a 0.22 μm organic filter membrane, put into an injection vial (including an inner tube), to obtain the test solution for use. All solutions were stored at 4° C. until analysis.

### 2.2.4 Chromatographic Conditions

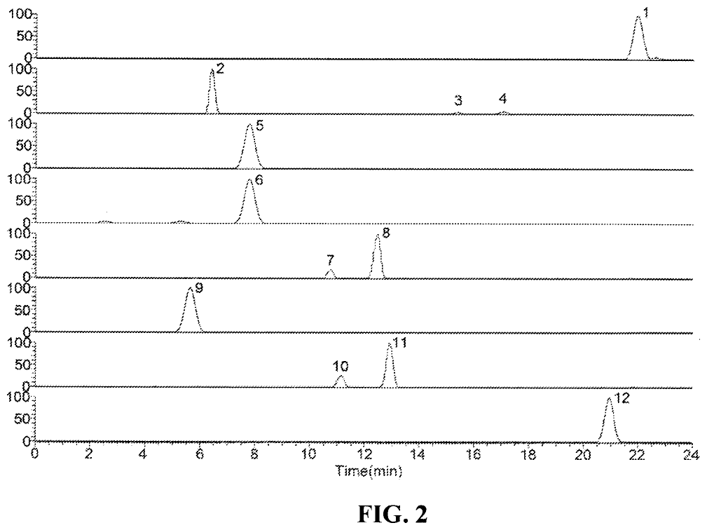

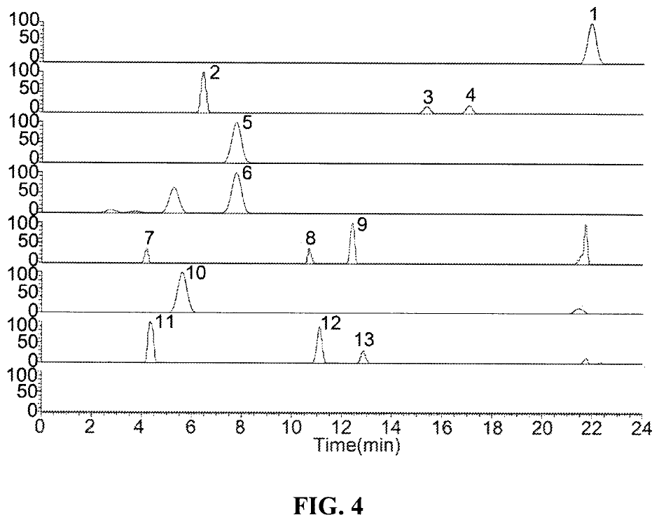

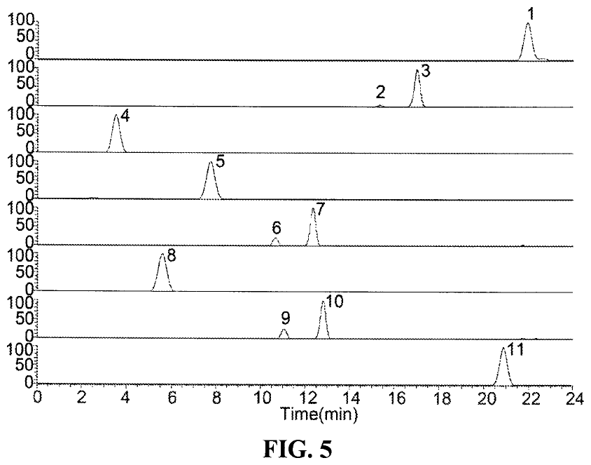

The chromatographic conditions were the same as “1.2.3 Chromatographic conditions”. The chromatograms of the samples were shown in FIG. 2 to FIG. 5.

### 2.2.5 MS Conditions

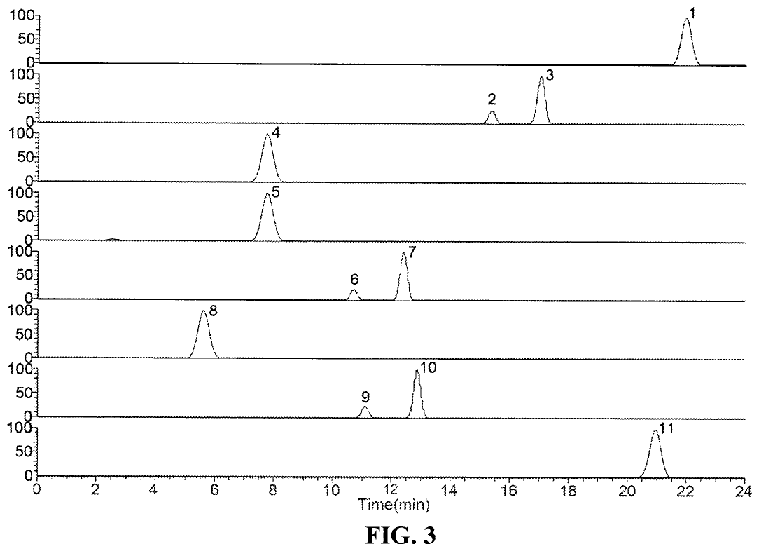

The MS conditions were the same as “1.2.4 MS conditions”. Taking a certain batch as an example, the PRM extracted ion chromatogram of the sample solutions of Calculus Bovis Artifactus, ox bile powder, Calculus Bovis, and Calculus Bovis Sativus were shown in FIG. 3 to FIG. 6, and the compound ion pairs, optimized collision energy (CE) and other information were shown in Table 1 under “1.2.4 MS conditions”.

### 2.2.6 Linearity, Limit of Detection (LOD), and Limit of Quantification (LOQ)

A series of mixed standard solutions with concentration gradients prepared under “2.2.2 Preparation of standard curve of mixed standard solution”, analyzed according to “2.2.4 Chromatographic conditions” and “2.2.5 MS conditions”, the chromatographic peak areas were recorded, and the linear ranges of the final determined standards were shown in Table 5. With the mass concentration of the standard as an abscissa (X) and the peak area ratio of the standard to be tested to the internal standard as an ordinate (Y), a standard curve was drawn and a linear regression was conducted. The amount of each standard when the signal-to-noise ratio (S/N)=3 was taken as LOD, and the amount of each standard when the signal-to-noise ratio (S/N)=10 was taken as LOQ. The results showed that the 17 components had desirable linear relationships in each concentration range, and the r² values were all greater than or equal to 0.9991. The regression equation, linear range, LOD, and LOQ of each component were shown in Table 3.

TABLE 3
Regression equation, correlation coefficient,
linear range, LOD, and LOQ of 17 CA components:

| | | | Linear Ranges | LOD | LOQ |
|---|---|---|---|---|---|
| Analyte | Regression equations | r² | (ng · mL⁻¹) | (ng · mL⁻¹) | (ng · mL⁻¹) |
| CDCA | Y = 4.296e⁻²X + 1.871e⁻² | 0.9994 | 0.5-200 | | |
| HDCA | Y = 4.311e⁻²X + 8.636e⁻² | 0.9995 | 0.5-1000 | 0.15 | 0.50 |
| UDCA | Y = 2.817e⁻²X − 2.065e⁻³ | 0.9998 | 0.5-200 | 0.15 | 0.50 |
| DCA | Y = 3.454e⁻²X + 8.045e⁻² | 0.9996 | 0.5-1000 | 0.15 | 0.50 |
| TUDCA | Y = 1.615e⁻²X − 4.792e⁻² | 0.9993 | 2-250 | 0.60 | 2.0 |
| TCDCA | Y = 1.556e⁻²X − 7.712e⁻² | 0.9996 | 2-1000 | 0.60 | 2.0 |
| THDCA | Y = 1.61e⁻²X − 2.8e⁻² | 0.9995 | 0.5-250 | 0.15 | 0.50 |
| TDCA | Y = 2.076e⁻²X − 7.555e⁻² | 0.9995 | 0.1-1000 | 0.03 | 0.10 |
| GCDCA | Y = 1.773e⁻²X − 4.437e⁻³ | 0.9998 | 0.5-200 | 0.15 | 0.50 |
| GUDCA | Y = 1.851e⁻²X − 3.378e⁻² | 0.9991 | 0.5-250 | 0.15 | 0.50 |
| GHDCA | Y = 1.548e⁻²X − 2.688e⁻³ | 0.9997 | 0.5-200 | 0.15 | 0.50 |
| GDCA | Y = 2.135e⁻²X − 1.414e⁻¹ | 0.9991 | 0.5-1000 | 0.15 | 0.50 |
| GCA | Y = 1.352e⁻⁴X − 1.092e⁻² | 1.000 | 200-5000 | 60.00 | 200.00 |
| TCA | Y = 1.331e⁻²X − 2.844e⁻¹ | 0.9994 | 0.5-5000 | 0.15 | 0.50 |
| CA | Y = 1.489e⁻²X + 7.79e⁻² | 0.9996 | 1-2000 | 0.30 | 1.00 |
| LCA | Y = 9.332e⁻²X + 9.461e⁻² | 0.9997 | 0.1-1000 | 0.03 | 0.10 |
| TLCA | Y = 1.902e⁻²X − 3.939e⁻² | 0.9992 | 0.5-250 | 0.15 | 0.50 |

### 2.2.7 Precision Experiment

The samples of mixed Calculus Bovis and substitutes thereof were taken and used to prepare the QC sample solution according to the method under “2.2.3 Preparation of test solutions”. the sample was injected 6 times continuously according to “2.2.4 Chromatographic conditions” and “2.2.5 MS conditions”, the peak area of the quality control (QC) sample solution was recorded, and the results showed that the RSD values were all less than 4.90%, indicating that the instrument had high precision. The specifically information was shown in Table 4.

### 2.2.8 Stability Experiment

The mixed Calculus Bovis samples and substitutes thereof were taken and used to prepare QC sample solution according to the method under “2.2.3 Preparation of test solutions”, and sample injection and analysis were conducted at 0, 4, 8, 12, 18, and 24 h according to “2.2.4 Chromatographic conditions” and “2.2.5 MS conditions”, the peak area was recorded to examine the stability of the test solution. The results showed that the RSD values were all less than 4.80%, indicating that the test solution was stable within 24 h. The specifically information was shown in Table 4.

### 2.2.9 Reproducibility Test

6 samples of mixed Calculus Bovis and substitutes thereof were accurately weighed and used to prepare the QC sample solution according to the method under “2.2.3 Preparation of test solutions”, the sample was detected according to “2.2.4 Chromatographic conditions” and “2.2.5 MS conditions”, the peak area of the QC sample solution was recorded to calculate the content, and the results showed that the RSD values were all less than 4.20%, indicating that there was desirable reproducibility. The specifically information was shown in Table 4.

TABLE 4
Precision, reproducibility, and stability of 17 CA components

| | Precision | Repeatability | Stability (RSD, %) |
|---|---|---|---|
| | (RSD, %) | (RSD, %) | (n = 6, 0, 4, 8, |
| Analyte | n = 6 | n = 6 | 12, 18, 24 h) |
| CDCA | 4.49 | 1.67 | 3.40 |
| HDCA | 1.06 | 1.53 | 0.13 |
| UDCA | 3.75 | 4.15 | 4.16 |
| DCA | 3.17 | 2.00 | 3.62 |
| TUDCA | — | — | 0.06 |
| TCDCA | 1.25 | 1.30 | 1.36 |
| THDCA | 4.38 | 0.28 | 1.68 |
| TDCA | 2.37 | 0.91 | 2.04 |
| GCDCA | 0.73 | 3.23 | 0.58 |
| GUDCA | 2.35 | 1.76 | — |
| GHDCA | 4.68 | 2.04 | 4.76 |
| GDCA | 3.93 | 2.13 | 4.52 |
| GCA | — | — | — |
| TCA | 2.22 | 3.52 | 1.17 |
| CA | 4.87 | 1.83 | 3.16 |
| LCA | 3.64 | 2.54 | 3.99 |
| TLCA | 3.35 | 3.78 | 3.60 |

### 2.2.10 Sample Determination

5 mg of 8 batches of Calculus Bovis Artifactus, 8 batches of ox bile powder, 8 batches of Calculus Bovis, and 8 batches of Calculus Bovis Sativus samples were taken and accurately weighed. The test solution was prepared according to the method in “2.2.3 Preparation of test solutions”. The determination was conducted according to the analytical conditions in “2.2.4 Chromatographic conditions” and “2.2.5 MS conditions”. The corresponding peak areas of different CA components were recorded. According to the internal standard method, the peak area ratios of 17 analytes and internal standards were substituted into the corresponding regression equation to calculate their mass fractions. The results were shown in Table 5. As shown in Table 5, the content of CA in Calculus Bovis was significantly lower than that in the other three categories, among which CA was the most obvious, and DCA, TCDCA, TDCA, GDCA, and TCA were relatively obvious. Calculus Bovis Artifactus contained more HDCA, while the other three categories did not contain HDCA. Calculus Bovis Sativus contained more DCA. Ox bile powder contained more TCDCA, TDCA, GCDCA, GDCA, and TCA, and could be distinguished from the other three categories. In addition, the four types of Calculus Bovis and their substitutes did not contain TUDCA, and most of them did not contain THDCA, GUDCA, and GCA.

TABLE 5
Mass fractions of 17 CA components in Calculus Bovis Artifactus, ox bile powder, Calculus Bovis,
and Calculus Bovis Sativus (unit: mg g⁻¹, ND meant not detected or below LOD)

| Sample | | | | | | | | | |
|---|---|---|---|---|---|---|---|---|---|
| No. | CDCA | HDCA | UDCA | DCA | TUDCA | TCDCA | THDCA | TDCA | GCDCA |
| A-1 | 3.33 | 73.45 | 0.26 | 6.61 | ND | 2.48 | ND | 6.61 | 2.35 |
| A-2 | 3.85 | 83.57 | 0.31 | 7.58 | ND | 2.28 | 0.36 | 6.18 | 2.60 |
| A-3 | 2.92 | 64.47 | 0.23 | 5.74 | ND | 2.22 | ND | 6.31 | 2.11 |
| A-4 | 3.88 | 83.37 | 0.31 | 7.50 | ND | 2.71 | ND | 7.83 | 2.84 |
| A-5 | 1.51 | 74.55 | 0.36 | 7.59 | ND | 2.97 | ND | 7.41 | 1.89 |
| A-6 | 1.38 | 67.96 | 0.37 | 7.26 | ND | 2.50 | ND | 6.30 | 1.69 |
| A-7 | 1.04 | 50.70 | 0.24 | 5.22 | ND | 2.47 | ND | 6.43 | 1.31 |
| A-8 | 1.40 | 65.68 | 0.31 | 6.84 | ND | 2.68 | ND | 7.37 | 1.63 |
| B-1 | 2.22 | ND | 0.26 | 6.79 | ND | 9.73 | ND | 40.78 | 22.14 |
| B-2 | 2.14 | ND | 0.31 | 6.69 | ND | 8.65 | ND | 35.43 | 21.45 |
| B-3 | 1.76 | ND | 0.23 | 5.69 | ND | 8.88 | ND | 33.58 | 19.05 |
| B-4 | 2.04 | ND | 0.31 | 6.40 | ND | 8.71 | ND | 35.57 | 21.38 |
| B-5 | 3.21 | ND | 0.36 | 15.26 | ND | 14.30 | ND | 49.81 | 8.22 |
| B-6 | 3.01 | ND | 0.37 | 14.08 | ND | 13.09 | ND | 45.32 | 7.78 |
| B-7 | 3.05 | ND | 0.24 | 14.00 | ND | 13.15 | ND | 42.71 | 7.74 |
| B-8 | 3.29 | ND | 0.31 | 15.72 | ND | 14.64 | ND | 50.09 | 8.12 |
| C-1 | 4.69 | ND | 0.38 | 6.35 | ND | ND | 0.38 | 0.82 | 1.41 |
| C-2 | 4.15 | ND | 0.26 | 0.92 | ND | ND | 0.38 | 0.75 | 2.02 |
| C-3 | 2.51 | ND | 0.16 | 2.16 | ND | 1.14 | ND | 0.89 | 1.26 |
| C-4 | 3.34 | ND | 0.22 | 1.92 | ND | ND | ND | 0.77 | 1.27 |
| C-5 | 0.05 | ND | 0.07 | ND | ND | ND | ND | 0.75 | 0.11 |
| C-6 | 0.07 | ND | 0.09 | ND | ND | ND | ND | 0.76 | 0.21 |
| C-7 | 0.11 | ND | 0.06 | ND | ND | ND | ND | 0.75 | 0.61 |
| C-8 | 0.10 | ND | 0.08 | ND | ND | ND | ND | 0.78 | 0.54 |
| D-1 | 1.91 | ND | 0.18 | 43.08 | ND | 2.73 | 0.51 | 6.80 | 2.21 |
| D-2 | 0.96 | ND | 0.30 | 42.40 | ND | 2.60 | ND | 6.78 | 2.01 |
| D-3 | 0.81 | ND | 0.31 | 43.06 | ND | 2.51 | ND | 6.59 | 2.22 |
| D-4 | 1.04 | ND | 0.17 | 34.10 | ND | 3.14 | ND | 8.59 | 2.30 |
| D-5 | 3.39 | ND | 0.34 | 46.17 | ND | 3.99 | ND | 10.43 | 2.27 |
| D-6 | 2.22 | ND | 0.29 | 33.52 | ND | 2.78 | ND | 7.88 | 2.20 |
| D-7 | 1.01 | ND | 0.15 | 33.76 | ND | 3.14 | ND | 8.50 | 2.30 |
| D-8 | 3.62 | ND | 0.41 | 45.86 | ND | 3.39 | ND | 8.60 | 2.17 |

| Sample | | | | | | | | |
|---|---|---|---|---|---|---|---|---|
| No. | GUDCA | GHDCA | GDCA | GCA | TCA | CA | LCA | TLCA |
| A-1 | ND | 0.06 | 9.12 | ND | 33.00 | 89.65 | 0.03 | ND |
| A-2 | ND | 0.09 | 10.20 | ND | 29.28 | 99.13 | 0.04 | ND |
| A-3 | ND | 0.10 | 8.25 | ND | 27.85 | 75.14 | 0.01 | ND |
| A-4 | ND | 0.16 | 10.45 | ND | 35.21 | 101.61 | 0.03 | 0.46 |
| A-5 | ND | 0.11 | 7.45 | ND | 46.89 | 91.62 | 0.01 | ND |
| A-6 | ND | 0.07 | 6.78 | ND | 37.33 | 88.13 | ND | 0.48 |
| A-7 | ND | 0.16 | 5.77 | ND | 36.99 | 68.03 | ND | ND |
| A-8 | ND | 0.06 | 6.97 | ND | 45.86 | 84.34 | 0.59 | ND |
| B-1 | 0.57 | 0.04 | 100.15 | ND | 179.87 | 53.76 | ND | 1.07 |
| B-2 | 0.64 | 0.10 | 94.56 | ND | 170.22 | 49.52 | ND | 1.13 |
| B-3 | 0.51 | 0.08 | 83.17 | ND | 153.68 | 45.66 | ND | 0.94 |
| B-4 | 0.49 | 0.04 | 94.78 | ND | 169.15 | 49.32 | ND | 1.00 |
| B-5 | ND | ND | 28.67 | ND | 316.23 | 129.96 | 0.02 | 1.05 |
| B-6 | ND | ND | 27.69 | ND | 272.11 | 124.11 | ND | 0.94 |
| B-7 | ND | ND | 27.84 | ND | 268.77 | 127.07 | ND | 0.77 |
| B-8 | ND | ND | 28.03 | ND | 323.76 | 129.89 | 0.03 | 1.00 |
| C-1 | ND | 1.81 | 1.73 | ND | 5.03 | 12.86 | 42.11 | ND |
| C-2 | ND | 2.19 | 1.50 | 57.02 | 4.74 | 3.19 | 28.67 | ND |
| C-3 | ND | 0.90 | 1.68 | ND | 5.56 | 4.59 | 20.98 | ND |
| C-4 | ND | 1.29 | 1.55 | ND | 4.65 | 4.65 | 26.22 | ND |
| C-5 | ND | ND | 1.38 | ND | 4.51 | ND | ND | ND |
| C-6 | ND | ND | 1.40 | ND | 4.75 | ND | ND | ND |
| C-7 | ND | ND | 1.52 | ND | 4.59 | ND | ND | ND |
| C-8 | ND | ND | 1.53 | ND | 4.83 | ND | ND | ND |
| D-1 | ND | ND | 8.70 | ND | 34.32 | 140.81 | 0.01 | 0.50 |
| D-2 | ND | ND | 8.73 | ND | 32.16 | 126.09 | ND | 0.47 |
| D-3 | ND | ND | 9.10 | ND | 32.04 | 125.24 | ND | 0.45 |
| D-4 | ND | ND | 9.45 | ND | 42.16 | 130.38 | ND | ND |
| D-5 | ND | ND | 8.90 | ND | 52.07 | 150.63 | 0.02 | 0.55 |
| D-6 | ND | 0.04 | 9.15 | ND | 38.98 | 153.54 | 0.10 | ND |
| D-7 | ND | ND | 9.58 | ND | 40.59 | 127.79 | ND | 0.54 |
| D-8 | ND | ND | 8.53 | ND | 39.96 | 155.74 | 0.09 | ND |

### 2.2.11 Optimization of Chromatographic Conditions

Since there were many types of CA components for detection and their chemical structures were similar, some of them were isomers with the same detection ion pairs, and could not be identified by mass spectrometry. Therefore, these CA components must be completely separated in the liquid phase for qualitative and quantitative analysis. This experiment investigated the resolution of 0.1% formic acid water, 5 mmol·L⁻¹ ammonium acetate solution, and pure water as mobile phases, as well as the separation effects of Waters CORTECS C18 (4.6×50 mm, 2.7 μm) and Hypersil Gold C18 (100×2.1 mm, 1.9 μm) columns. Finally, the Hypersil Gold C18 column (100×2.1 mm, 1.9 μm) was selected based on better separation effects, 5 mmol·L⁻¹ ammonium acetate solution as mobile phase A, and methanol solution as mobile phase B, with better peak shape and higher response. In addition, since the CA components were weakly acidic, the negative ion mode was selected to collect the mass spectrometry data of CA components.

### 3. Analysis of Significant Difference Components

### 3.1 HCA

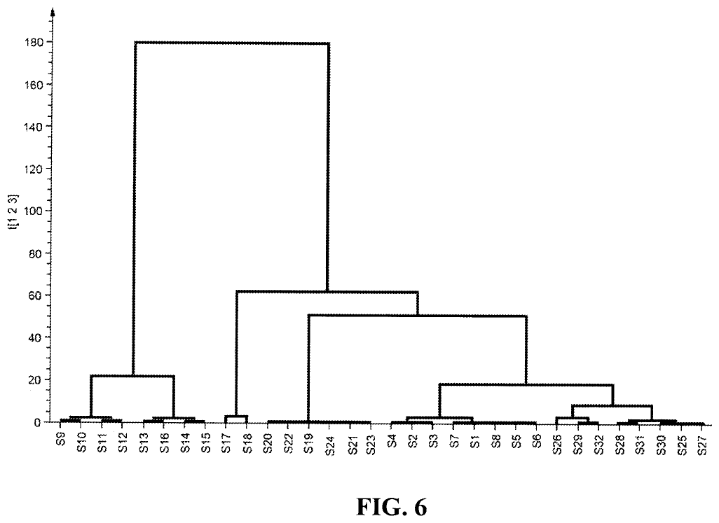

HCA is a multivariate analysis technique that can divide samples according to their internal characteristics. In this study, SIMCA14.1 software was used to allow HCA, and the similarity measure was calculated by the square of hierarchical distance and Euclidean distance. The degree of correlation between samples depends on the distance of the tree. The shortest distance indicated a height of the relationship. Therefore, these objects were considered to be attributes of the same group. This figure showed a dendrogram used as a tool to explain the closest distances between sample clusters. By classifying different batches of Calculus Bovis samples, statistically significant quality evaluation indicators were selected. The HCA results of Calculus Bovis showed that the 32 batches of Calculus Bovis-like substances were divided into four main clusters, Calculus Bovis Artifactus was divided into group I, and S1, S2, S3, S4, S5, S6, and S7 were divided into one group. Ox bile powder was divided into group II, with S8, S9, and S10 to S16 as one group. Calculus Bovis was divided into group III, with S17 to S24 as one group. The Calculus Bovis Sativus was divided into group IV, with S25 to S32 as one group, and the results were shown in FIG. 6.

### 3.2 PCA

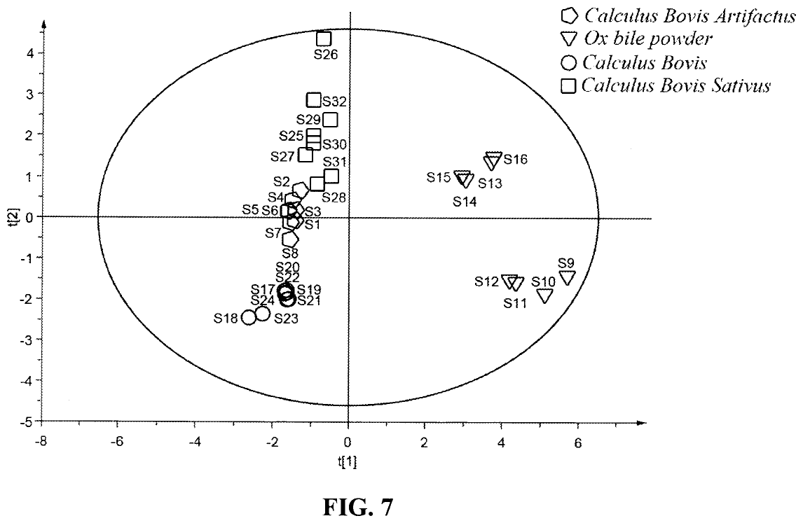

PCA was conducted to solve the problem of multicollinearity and explore the relationship between independent variables. In addition, whether the differences in CA types and their contents could effectively group different types of samples should to be evaluated. In PCA, a large amount of data was replaced by principal component score (PCS), which contained the same information as the original data. The PCA was generated by calculating the average relative peak area of the common peak centers of the samples of Calculus Bovis and substitutes thereof. The first principal component (PC1) contained the largest variance in the data, and the second principal component (PC2) represented the largest variance that was not explained by PC1. According to the PCA score plot of Calculus Bovis and substitutes thereof (FIG. 7), the top two PCs, PC1 and PC2, accounted for 44.2% and 28.7% of the total variance of the original observations, respectively, and 74.1% of the total variance. As shown in the PCA score plot of Calculus Bovis and substitutes thereof (FIG. 7), they could be divided into four groups according to the different sources of samples. S1 to S8 could be easily grouped together, with SI being found near S2 to S7, and their chemical compositions were related to each other. S1, S2, S3, S4, S5, S6, and S7 tended to be grouped into the same cluster based on the similarity of chemical composition. S9 to S16 could be easily grouped together, and their chemical compositions were related to each other. S10 was found next to S9; therefore, based on the similarity of chemical composition, S9, S10, S11, S12, S13, S14, S15, and S16 tended to be grouped in the same cluster. And so on, S17 to S24 were as one group; S25 to S32 were as one group.

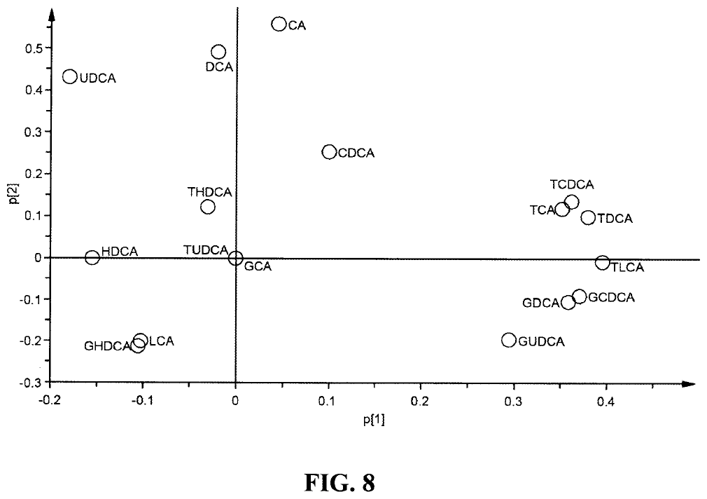

As shown in FIG. 8, the PCA loading diagram was of great significance for the identification of the types of 32 batches of Calculus Bovis and substitutes thereof. The loading diagram showed the multivariate variables that could influence the differences between samples. It can be seen that the differences in the contents of these common CA components had a certain impact on the identification of Calculus Bovis species. Each point in the PCA loading diagram of Calculus Bovis and substitutes thereof (FIG. 8) represented a CA component. The farther the point was from the origin, the greater the weight was, and the greater the role it played in distinguishing samples. As shown in FIG. 8, the two CA components TUDCA and GCA had little effect on distinguishing Calculus Bovis and substitutes thereof. The remaining CA components such as CA, DCA, HDCA, TCDCA, TDCA, TLCA, and GCDCA could contribute greatly to the differentiation of Calculus Bovis and substitutes thereof, and could be used as marker compounds for identifying Calculus Bovis and substitutes thereof.

### 3.3 OPLS-DA

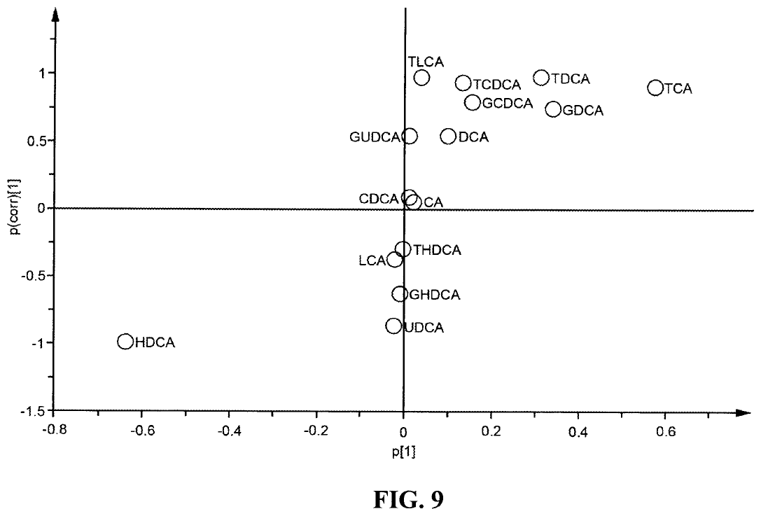

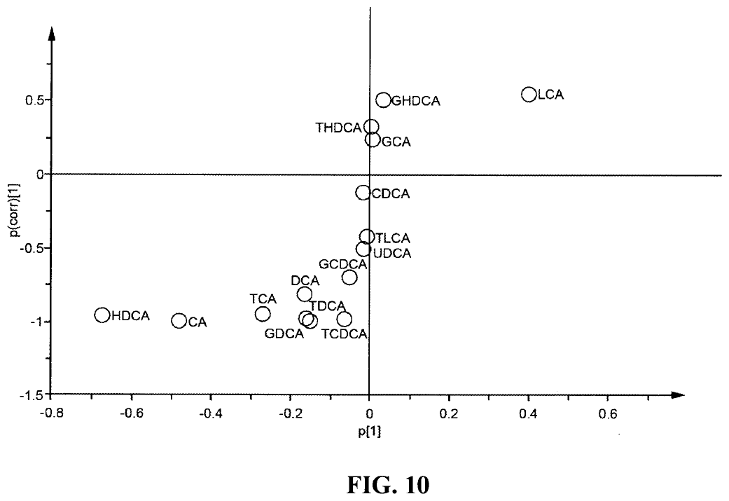

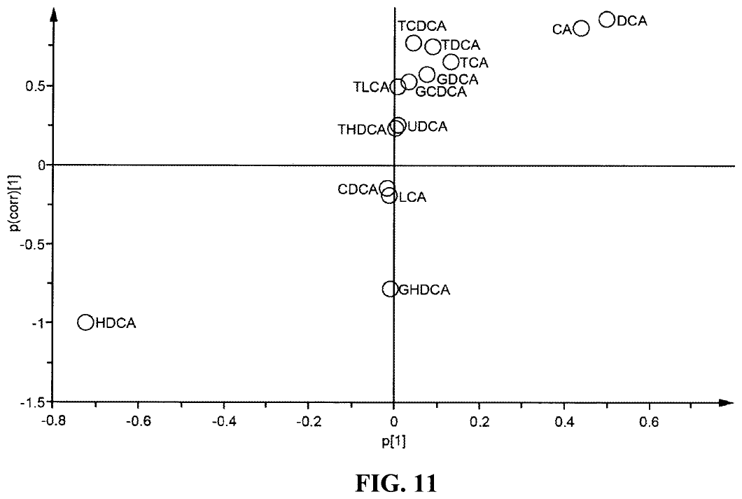

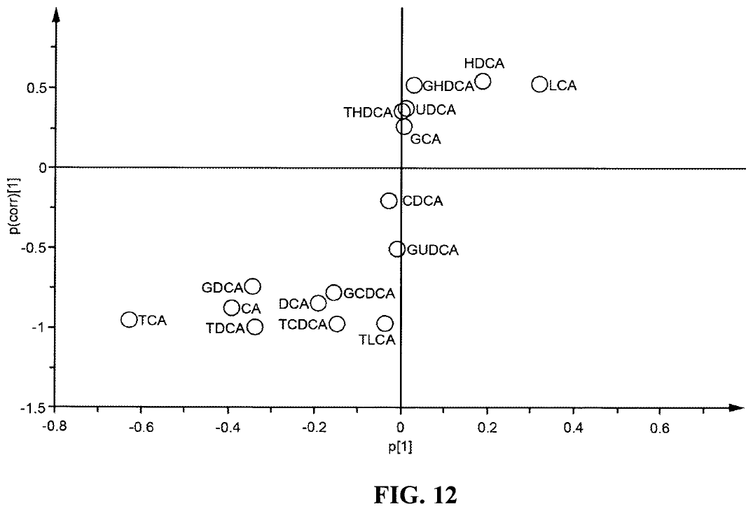

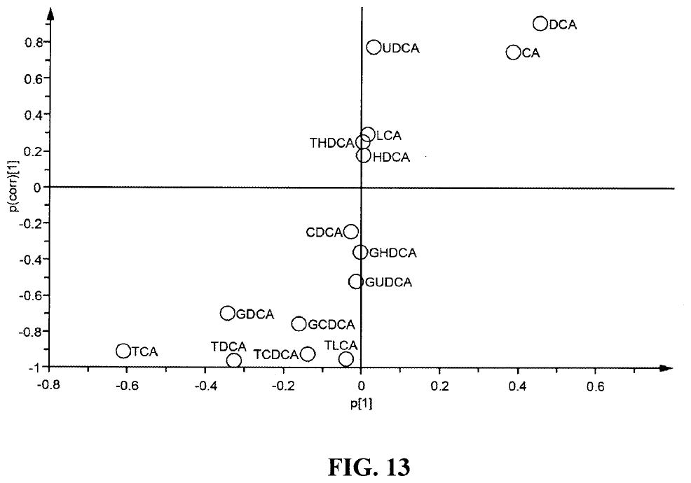

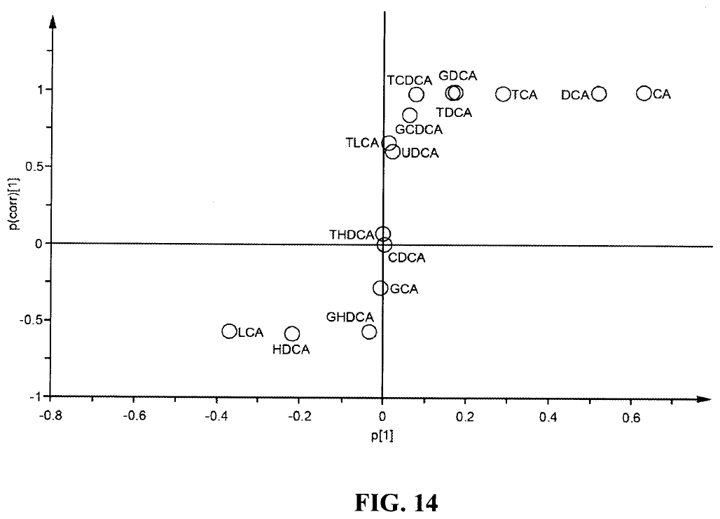

OPLS-DA was a supervised classification technique that improved interpretability. In view of the differences between Calculus Bovis and substitutes thereof, in order to identify Calculus Bovis and substitutes thereof more accurately, S-Plot diagrams were made for comparison of cach two of the Calculus Bovis and substitutes thereof to find out the CA components with the largest difference. Since the CA content of Calculus Bovis and substitutes thereof was different, when the cach two were compared, there were not necessarily exactly 17 CA components. There were differences in the content and types of CA between two different types of Calculus Bovis and substitutes thereof, as shown in FIG. 9 to FIG. 14. From the S-Plot diagram of the CA components of Calculus Bovis Artifactus and ox bile powder (FIG. 9), it was scen that HDCA, TDCA, TLCA, GCDCA, had the greatest influence on the identification of Calculus Bovis Artifactus and ox bile powder. From the S-Plot diagram of the CA components of Calculus Bovis Artifactus and Calculus Bovis (FIG. 10), HDCA, CA, LCA, GHDCA, and TDCA had the greatest influence on the identification of Calculus Bovis Artifactus and Calculus Bovis. From the S-Plot diagram of the CA components of Calculus Bovis Artifactus and Calculus Bovis Sativus (FIG. 11), it was seen that HDCA, DCA, CA, and TDCA had the greatest influence on the identification of Calculus Bovis Artifactus and Calculus Bovis Sativus. From the S-Plot diagram of the CA component of ox bile powder and Calculus Bovis (FIG. 12), it was seen that HDCA, DCA, CA, TDCA, GCDCA, and GHDCA had the greatest influence on the identification of ox bile powder and Calculus Bovis. From the S-Plot diagram of the CA components of ox bile powder and Calculus Bovis Sativus (FIG. 13), it was seen that DCA, CA, TDCA, TCA, TLCA, GCDCA, and UDCA had the greatest influence on the identification of ox bile powder and Calculus Bovis Sativus. From the S-Plot diagram of CA components loaded in Calculus Bovis and Calculus Bovis Sativus (FIG. 14), it was seen that DCA, CA, TDCA, HDCA, TLCA, and GCDCA had the greatest influence on the identification of Calculus Bovis and Calculus Bovis Sativus. Then, based on statistical data, 6 main CA components were screened out from Calculus Bovis and substitutes thereof. Finally, combining HCA, PCA, and OPLS-DA, it was seen that CA, TDCA, TLCA, GCDCA, HDCA, and DCA had the greatest impact on the identification of Calculus Bovis and substitutes thereof. The results of this study clearly showed that CA, TDCA, TLCA, GCDCA, HDCA, and DCA played a vital role in the identification of Calculus Bovis and substitutes thereof.

### 4 Establishment and Verification of Discriminant Analysis Equation

### 4.1 Establishment of Discriminant Analysis Equation

Discriminant analysis is an MVSA method that determines the category of the research object based on the various characteristic values under the condition of a certain classification. In this study, a domain U={X1, X2, . . . , X32} representing 32 batches of Calculus Bovis and substitutes thereof was established, and the original contents of 6 characteristic CA components, including CA, TDCA, TLCA, GCDCA, HDCA, and DCA (the original concentration data calculated by the instrument, unit: ng·mL⁻¹) were selected as discriminant factors to form a 32×6matrix, as shown in formula (1).

Xi = (Xil, Xi2, . . . , Xi6)

In the formula, i represented the number of samples; X represented the sample.

Stepwise discriminant analysis was conducted using SPSS26.0 software and Wilk's lambda method to obtain the discriminant function for determining the types of Calculus Bovis and substitutes thereof. The quality of the discriminant function was evaluated by regression estimation method. Finally, the discriminant function equations of Calculus Bovis and substitutes were obtained as follows (S1: CA, S2: TDCA, S3: TLCA, S4: GCDCA, S5: HDCA, S6: DCA):

$$Y 1 = - 0.0 \; 34 S 1 + 0.3 \; 45 S 2 - 3.071 S 3 + 0.1 \; 21 S 4 + 0.5 \; 15 S 5 + 0.0 \; 71 S 6 - 91.575 ( Calculus Bovis Artifactus )$$

$$Y 2 = 0.2 \; 53 S 1 + 2.1 \; 42 S 2 + 4.9 \; 59 S 3 + 3.1 \; 61 S 4 - 0.446 S 5 - 0.207 S 6 - 401.886 ( ox bile powder )$$

$$Y 3 = 0. 0 \; 15 S 1 + 0.0 \; 37 S 2 - 0.112 S 3 + 0.1 \; 45 S 4 - 0.023 S 5 + 0.0 \; 23 S 6 - 1.997 ( Calculus Bovis )$$

$$Y 4 = 0. 4 \; 72 S 1 - 0.783 S 2 + 5.0 \; 61 S 3 + 2.3 \; 61 S 4 - 0.619 S 5 + 0.3 \; 79 S 6 - 204.836 ( Calculus Bovis Sativus ) .$$

### 4.2 Verification of Discriminant Analysis Equation

### 4.2.1 Verification Method

A total of 23 batches of experimental samples were collected, and the specific sample information was recorded in Table 6. 6 batches of Calculus Bovis Artifactus and 5 batches of ox bile powder were provided by Wuhan Baicaoyuan Biochemical Pharmaceutical Co., Ltd.; 6 batches of Calculus Bovis were provided by Guangdong Medicinal Materials Company; 6 batches of Calculus Bovis Sativus were provided by Wuhan Jianmin Dapeng Pharmaceutical Co., Ltd. 5 mg of each batch of samples were accurately weighed to prepare mixed standard solutions according to the method in “2.2 Methods and results”. The test solutions were measured under the analytical conditions in that item and the original concentration data calculated by the instrument were collated, as shown in Table 7.

TABLE 6
Sample information

| Number | Sample | Batch | Source |
|---|---|---|---|
| A-9 | Calculus Bovis | 210303 | Wuhan Baicaoyuan Biochemical |
| | Artifactus | | Pharmaceutical Co., Ltd. |
| A-10 | Calculus Bovis | 210103 | Wuhan Baicaoyuan Biochemical |
| | Artifactus | | Pharmaceutical Co., Ltd. |
| A-11 | Calculus Bovis | 210304 | Wuhan Baicaoyuan Biochemical |
| | Artifactus | | Pharmaceutical Co., Ltd. |
| A-12 | Calculus Bovis | 210104 | Wuhan Baicaoyuan Biochemical |
| | Artifactus | | Pharmaceutical Co., Ltd. |
| A-13 | Calculus Bovis | 210101 | Wuhan Baicaoyuan Biochemical |
| | Artifactus | | Pharmaceutical Co., Ltd. |
| A-14 | Calculus Bovis | 210102 | Wuhan Baicaoyuan Biochemical |
| | Artifactus | | Pharmaceutical Co., Ltd. |
| B-9 | Ox bile powder | B210102-1 | Wuhan Baicaoyuan Biochemical |
| | | | Pharmaceutical Co., Ltd. |
| B-10 | Ox bile powder | B210102-2 | Wuhan Baicaoyuan Biochemical |
| | | | Pharmaceutical Co., Ltd. |
| B-11 | Ox bile powder | B201101-1 | Wuhan Baicaoyuan Biochemical |
| | | | Pharmaceutical Co., Ltd. |
| B-12 | Ox bile powder | B210101-1 | Wuhan Baicaoyuan Biochemical |
| | | | Pharmaceutical Co., Ltd. |
| B-13 | Ox bile powder | B210101-2 | Wuhan Baicaoyuan Biochemical |
| | | | Pharmaceutical Co., Ltd. |
| C-9 | Calculus Bovis | 201105-5 | Guangdong Medicinal |
| | | | Materials Company |
| C-10 | Calculus Bovis | 201105-6 | Guangdong Medicinal |
| | | | Materials Company |
| C-11 | Calculus Bovis | 201105-7 | Guangdong Medicinal |
| | | | Materials Company |
| C-12 | Calculus Bovis | 201105-8 | Guangdong Medicinal |
| | | | Materials Company |
| C-13 | Calculus Bovis | 201105-9 | Guangdong Medicinal |
| | | | Materials Company |
| C-14 | Calculus Bovis | 201105-10 | Guangdong Medicinal |
| | | | Materials Company |
| D-9 | Calculus Bovis | 190303 | Wuhan Jianmin Dapeng |
| | Sativus | | Pharmaceutical Co., Ltd. |
| D-10 | Calculus Bovis | 191202 | Wuhan Jianmin Dapeng |
| | Sativus | | Pharmaceutical Co., Ltd. |
| D-11 | Calculus Bovis | 200610 | Wuhan Jianmin Dapeng |
| | Sativus | | Pharmaceutical Co., Ltd. |
| D-12 | Calculus Bovis | 191203 | Wuhan Jianmin Dapeng |
| | Sativus | | Pharmaceutical Co., Ltd. |
| D-13 | Calculus Bovis | 201002 | Wuhan Jianmin Dapeng |
| | Sativus | | Pharmaceutical Co., Ltd. |
| D-14 | Calculus Bovis | 190712 | Wuhan Jianmin Dapeng |
| | Sativus | | Pharmaceutical Co., Ltd. |

TABLE 7
Original content of 17 CA components in Calculus Bovis Artifactus, ox bile powder, Calculus Bovis,
and Calculus Bovis Sativus (unit: ng mL⁻¹, ND meant not detected or below LOD)

| Sample | | | | | | | | | |
|---|---|---|---|---|---|---|---|---|---|
| No. | CDCA | HDCA | UDCA | DCA | TUDCA | TCDCA | THDCA | TDCA | GCDCA |
| A-9 | 7.56 | 356.73 | 1.66 | 37.44 | ND | 13.28 | ND | 34.14 | 9.03 |
| A-10 | 7.30 | 337.99 | 1.87 | 36.47 | ND | 12.78 | ND | 32.08 | 9.10 |
| A-11 | 7.87 | 364.66 | 1.73 | 39.48 | ND | 19.95 | ND | 59.23 | 13.59 |
| A-12 | 6.99 | 326.88 | 1.55 | 32.76 | ND | 14.65 | ND | 40.23 | 8.08 |
| A-13 | 7.51 | 378.43 | 1.64 | 37.59 | ND | 18.21 | ND | 54.68 | 9.23 |
| A-14 | 8.39 | 389.71 | 2.07 | 42.51 | ND | 13.78 | ND | 36.99 | 9.94 |
| B-9 | 15.74 | ND | 0.70 | 71.28 | ND | 69.20 | ND | 234.65 | 38.67 |
| B-10 | 15.81 | ND | 0.91 | 72.21 | ND | 63.87 | ND | 207.49 | 37.76 |
| B-11 | 15.61 | ND | 0.96 | 74.28 | ND | 70.75 | ND | 240.75 | 40.36 |
| B-12 | 15.51 | ND | 0.82 | 70.85 | ND | 67.95 | ND | 229.87 | 40.28 |
| B-13 | 13.30 | ND | 0.70 | 65.75 | ND | 62.23 | ND | 203.84 | 35.66 |
| C-9 | 0.18 | ND | 0.28 | ND | ND | ND | ND | 4.01 | 1.29 |
| C-10 | 0.27 | ND | 0.35 | ND | ND | ND | ND | 3.67 | 0.81 |
| C-11 | 0.35 | ND | 0.46 | ND | ND | ND | ND | ND | 0.38 |
| C-12 | 0.67 | ND | 0.90 | ND | ND | ND | ND | ND | 2.54 |
| C-13 | 0.91 | ND | 0.70 | ND | ND | ND | ND | 3.69 | 2.45 |
| C-14 | 0.73 | ND | 1.17 | ND | ND | ND | ND | 3.67 | 0.50 |
| D-9 | 10.08 | ND | 1.31 | 153.42 | ND | 13.25 | ND | 37.12 | 10.26 |
| D-10 | 18.46 | ND | 1.88 | 226.71 | ND | 14.41 | ND | 34.10 | 10.21 |
| D-11 | 10.82 | ND | 1.07 | 228.24 | ND | 10.79 | ND | 28.54 | 10.10 |
| D-12 | 19.28 | ND | 2.16 | 243.64 | ND | 13.79 | ND | 32.28 | 11.53 |
| D-13 | 8.41 | ND | 0.95 | 237.07 | ND | 12.91 | ND | 32.34 | 12.95 |
| D-14 | 13.05 | ND | 2.07 | 312.75 | ND | 24.09 | ND | 67.61 | 19.56 |

| Sample | | | | | | | | |
|---|---|---|---|---|---|---|---|---|
| No. | GUDCA | GHDCA | GDCA | GCA | TCA | CA | LCA | TLCA |
| A-9 | ND | 0.28 | 36.36 | ND | 200.88 | 461.43 | ND | ND |
| A-10 | ND | 0.52 | 35.26 | ND | 205.38 | 444.33 | ND | ND |
| A-11 | ND | 0.62 | 62.78 | ND | 356.75 | 485.29 | ND | 2.54 |
| A-12 | ND | 0.43 | 35.09 | ND | 235.83 | 418.36 | ND | ND |
| A-13 | ND | 0.45 | 36.63 | ND | 317.60 | 460.77 | ND | 2.60 |
| A-14 | ND | 0.78 | 40.11 | ND | 233.13 | 504.54 | ND | ND |
| B-9 | ND | ND | 139.14 | ND | 1411.96 | 618.69 | ND | 5.05 |
| B-10 | ND | ND | 134.58 | ND | 1304.50 | 641.98 | ND | 4.21 |
| B-11 | ND | ND | 141.03 | ND | 1606.61 | 667.50 | ND | 5.26 |
| B-12 | ND | ND | 139.45 | ND | 1416.59 | 623.21 | ND | 4.88 |
| B-13 | ND | ND | 128.63 | ND | 1364.60 | 579.45 | ND | 4.20 |
| C-9 | ND | ND | 6.91 | ND | 21.96 | ND | ND | ND |
| C-10 | ND | ND | 6.83 | ND | 21.82 | ND | ND | ND |
| C-11 | ND | ND | ND | ND | 21.88 | ND | ND | ND |
| C-12 | ND | ND | 7.08 | ND | ND | ND | ND | ND |
| C-13 | ND | ND | 7.35 | ND | 23.22 | ND | ND | ND |
| C-14 | ND | ND | 6.78 | ND | 22.69 | ND | ND | ND |
| D-9 | ND | ND | 46.30 | ND | 174.50 | 796.49 | ND | 2.43 |
| D-10 | ND | ND | 39.59 | ND | 159.19 | 746.49 | 0.47 | 2.40 |
| D-11 | ND | ND | 45.34 | ND | 126.90 | 640.43 | 0.21 | ND |
| D-12 | ND | ND | 43.41 | ND | 155.21 | 808.10 | 0.65 | ND |
| D-13 | ND | ND | 48.80 | ND | 145.32 | 728.09 | ND | ND |
| D-14 | ND | 0.22 | 69.82 | ND | 319.35 | 1242.40 | ND | 2.75 |

### 4.2.2 Experimental Results

The corresponding contents of the 6 characteristic CA components after screening (Table 7) were substituted into the function equation, and the Y values of the function equations of different categories of Calculus Bovis and substitutes thereof were compared. Among them, the one with the maximum Y value belonged to the category of Calculus Bovis source represented by the equation. Through the regression estimation method, 5 batches of ox bile powder, 6 batches of Calculus Bovis Artifactus, 6 batches of Calculus Bovis, and 6 batches of Calculus Bovis Sativus of known categories were tested, and the source discrimination analysis of Calculus Bovis and substitutes thereof was compared with the actual results. The accuracy rate of ox bile powder was 100%, the accuracy rate of Calculus Bovis Artifactus was 100%, the accuracy rate of Calculus Bovis was 100%, and the accuracy rate of Calculus Bovis Sativus was 100% (Table 8). This indicated that the established discriminant function equation was relatively stable, could be used to predict and identify the source categories of Calculus Bovis and substitutes thereof, and showed values for promotion and application.

TABLE 8
Evaluation on effect for discriminant function equation
of Calculus Bovis and substitutes thereof

| | | Discriminant function predicts | | | |
|---|---|---|---|---|---|
| | | classification (source) | | | |
| | | Calculus | | | Calculus |
| | Original Calculus | Bovis | Ox bile | Calculus | Bovis |
| Statistics | Bovis categories | Artifactus | powder | Bovis | Sativus |
| Sample size | Calculus Bovis | 6 | 0 | 0 | 0 |
| | Artifactus | | | | |
| | Ox bile powder | 0 | 5 | 0 | 0 |
| | Calculus Bovis | 0 | 0 | 6 | 0 |
| | Calculus Bovis | 0 | 0 | 0 | 6 |
| | Sativus | | | | |
| Percentage | Calculus Bovis | 100 | 0 | 0 | 0 |
| (%) | Artifactus | | | | |
| | Ox bile powder | 0 | 100 | 0 | 0 |
| | Calculus Bovis | 0 | 0 | 100 | 0 |
| | Calculus Bovis | 0 | 0 | 0 | 100 |
| | Sativus | | | | |

### Example 2: Identification of Angong Niuhuang Wan containing Calculus Bovis or Calculus Bovis Sativus

500 mg each of Angong Niuhuang Wan samples containing Calculus Bovis from 7 batches and Angong Niuhuang Wan containing Angong Niuhuang Wan from 7 batches were taken, accurately weighed, and placed in 10 mL brown volumetric flasks. The prepared solvent (DMSO-CHCL₃—CH₃OH, 2:2:1, v/v, prepared and used immediately) was added to the scale, and the sealing film was tightly plugged. Ultrasonication (power of 250 W, frequency of 40 Hz) was conducted at 4° C. for 30 min in ice bath, and a resulting mixture was allowed to stand for 30 min, and then centrifuged (1,250 rpm, 10 min). The supernatant was taken and placed in a 10 mL EP tube. 100 μL of the supernatant was taken, diluted ten-fold and placed in a 2 mL EP tube to obtain a diluted sample. Then 100 μL of the diluted sample was taken and placed in a 2 mL EP tube, added with 100 μL of the internal standard solution prepared in “2.1.1 Preparation of standard stock solutions”, and then mixed with 800 μL of mass spectrometry-grade methanol to obtain a sample solution of 500 μg·mL⁻¹. The sample solution was centrifuged (1,250 rpm, 10 min), a resulting supernatant was taken, and filtered with a 0.22 μm organic filter membrane, put into an injection vial (including an inner tube), to obtain the test solution for use. All solutions were stored at 4° C. until analysis. The determination was conducted according to the analytical conditions in “2.2.4 Chromatographic conditions” and “2.2.5 MS conditions”. The corresponding peak areas of different CA components were recorded. According to the internal standard method, the peak area ratios of 17 analytes and internal standards were substituted into the corresponding regression equation to calculate their original concentrations by instruments. The results were shown in Table 9.

TABLE 9
Original concentrations of 17 CA components in 14 batches of different
varieties of Angong Niuhuang Wan (unit: ng · mL⁻¹)

| Sample | | | | | | | | | |
|---|---|---|---|---|---|---|---|---|---|
| No. | CDCA | HDCA | UDCA | DCA | TUDCA | TCDCA | THDCA | TDCA | GCDCA |
| CC-1 | 0.42 | ND | ND | 6.04 | ND | 0.21 | ND | 11.13 | ND |
| CC-2 | 2.38 | ND | ND | 9.77 | ND | 0.63 | ND | 9.88 | 1.07 |
| CC-3 | 0.14 | ND | ND | 4.70 | ND | 0.80 | ND | 11.93 | 0.21 |
| CC-4 | ND | ND | ND | 5.81 | ND | ND | ND | 9.43 | ND |
| CC-5 | ND | ND | ND | 3.81 | ND | ND | ND | 7.05 | ND |
| CC-6 | ND | ND | ND | 3.83 | ND | ND | ND | 8.57 | ND |
| CC-7 | 0.23 | ND | ND | 4.97 | ND | 0.43 | ND | 11.78 | ND |
| DD-1 | 39.96 | ND | 1.50 | 507.63 | 0.10 | 36.78 | ND | 137.76 | 17.11 |
| DD-2 | 34.92 | ND | 3.34 | 515.04 | ND | 41.73 | ND | 169.60 | 19.37 |
| DD-3 | 76.07 | ND | 7.06 | 1017.41 | 0.61 | 130.61 | ND | 480.57 | 48.99 |
| DD-4 | 41.86 | ND | 4.86 | 787.29 | 0.18 | 83.39 | ND | 282.60 | 28.71 |
| DD-5 | 43.36 | ND | 5.04 | 828.33 | 0.30 | 99.97 | ND | 330.11 | 42.80 |
| DD-6 | 30.73 | ND | 5.17 | 696.97 | 0.45 | 98.32 | ND | 313.40 | 41.98 |
| DD-7 | 18.17 | ND | 2.66 | 467.83 | 0.21 | 88.21 | ND | 272.05 | 40.50 |

| Sample | | | | | | | | |
|---|---|---|---|---|---|---|---|---|
| No. | GUDCA | GHDCA | GDCA | GCA | TCA | CA | LCA | TLCA |
| CC-1 | ND | ND | 1.80 | ND | 62.68 | ND | ND | ND |
| CC-2 | ND | ND | 4.60 | ND | 37.35 | 4.12 | ND | ND |
| CC-3 | ND | ND | 1.61 | ND | 63.87 | ND | ND | ND |
| CC-4 | ND | ND | 0.55 | ND | 50.92 | ND | ND | ND |
| CC-5 | ND | ND | ND | ND | 33.64 | ND | ND | ND |
| CC-6 | ND | ND | ND | ND | 43.69 | ND | ND | ND |
| CC-7 | ND | ND | 0.89 | ND | 65.29 | ND | ND | ND |
| DD-1 | ND | ND | 78.00 | ND | 703.40 | 2200.71 | 2.12 | ND |
| DD-2 | ND | ND | 100.33 | ND | 789.37 | 1809.96 | 1.79 | 0.06 |
| DD-3 | ND | ND | 225.15 | ND | 2493.29 | 4157.85 | 8.26 | 5.89 |
| DD-4 | ND | ND | 131.46 | ND | 1497.43 | 2685.51 | 5.01 | 1.67 |
| DD-5 | ND | ND | 197.36 | ND | 1676.08 | 2988.88 | 2.23 | 2.98 |
| DD-6 | 0.21 | ND | 208.84 | ND | 1551.78 | 2664.74 | 1.80 | 3.25 |
| DD-7 | ND | ND | 184.04 | ND | 1501.26 | 1902.95 | 0.80 | 1.40 |

The corresponding contents of the 6 characteristic CA components after screening by significant difference component analysis were substituted into the function equation, and the Y values of the function equations of Angong Niuhuang Wan containing Calculus Bovis and Angong Niuhuang Wan containing Calculus Bovis Sativus were compared. Among them, the one with the maximum Y value belonged to the category of Angong Niuhuang Wan source represented by the equation. Through the test of regression estimation method, 7 batches of Angong Niuhuang Wan containing Calculus Bovis and 7 batches of Angong Niuhuang Wan containing Calculus Bovis Sativus of known categories were tested, and the source discrimination analysis results of Angong Niuhuang Wan was compared with the facts. The accuracy rate of Angong Niuhuang Wan containing Calculus Bovis was 100%, and the accuracy rate of Angong Niuhuang Wan containing In vitro cultivated Calculus Bovis was 100% (Table 10). This indicated that the established discriminant function equation was relatively stable, and could not only realize the prediction and identification of the source categories of Calculus Bovis and substitutes thereof, but also realize the prediction and identification of the source categories of traditional Chinese medicines containing Calculus Bovis and substitutes thereof, such as Angong Niuhuang Wan containing Calculus Bovis or Calculus Bovis Sativus, which showed the values of promotion and application.

TABLE 10
Evaluation on effect for discriminant function equations of
14 batches of different varieties of Angong Niuhuang Wan

| | | Discriminant function predicts | |
|---|---|---|---|
| | | classification (source) | |
| | | | Angong |
| | | Angong | Niuhuang Wan |
| | | Niuhuang Wan | containing |
| | Original patent | containing | Calculus Bovis |
| Statistics | medicine categories | Calculus Bovis | Sativus |
| Sample size | Angong Niuhuang Wan | 7 | 0 |
| | containing Calculus | | |
| | Bovis | | |
| | Angong Niuhuang Wan | 0 | 7 |
| | containing Calculus | | |
| | Bovis Sativus | | |
| Percentage | Angong Niuhuang Wan | 100 | 0 |
| (%) | containing Calculus | | |
| | Bovis | | |
| | Angong Niuhuang Wan | 0 | 100 |
| | containing Calculus | | |
| | Bovis Sativus | | |

What is claimed is:

1. A method for identifying Calculus Bovis and a substitute thereof, comprising the following steps:
(1) subjecting a cholic acid (CA) component of the Calculus Bovis and the substitute thereof to qualitative and quantitative analysis by ultra-performance liquid chromatography-mixed quadrupole orbitrap mass spectrometry (UPLC-Q-orbitrap-MS), and then establishing a chromatogram of the CA component of the Calculus Bovis and the substitute thereof;
(2) screening a significantly different CA component in the Calculus Bovis and the substitute thereof by multivariate statistical analysis (MVSA); and
(3) establishing a discriminant function equation for the Calculus Bovis and the substitute thereof to identify the Calculus Bovis and the substitute thereof.

2. The method for identifying Calculus Bovis and a substitute thereof according to claim 1, wherein chromatographic conditions in step (1) comprise: a C18 chromatographic column; a mobile phase A of 5 mmol·L⁻¹ ammonium acetate solution, and a mobile phase B of methanol solution; a gradient elution program comprising: 60% B from 0 min to 2 min; 60% to 64% B from 2 min to 18 min; 64% to 95% B from 18 min to 19 min; 95% B from 19 min to 21 min; 95% to 60% B from 21 min to 22 min; and 60% B from 22 min to 24 min.

3. The method for identifying Calculus Bovis and a substitute thereof according to claim 1, wherein mass spectrometry conditions in step (1) comprise: an electrospray ionization (ESI) source, operation with a negative ion in a parallel reaction monitoring (PRM) mode, and a spray voltage of −3.8 kV; an ion source temperature of 380° C.; a sheath gas flow rate of 30 μL·min⁻¹; an auxiliary gas flow rate of 5 μL·min⁻¹; and an auxiliary gas N₂ heater temperature of 300° C.

4. The method for identifying Calculus Bovis and a substitute thereof according to claim 1, wherein the MVSA in step (2) is one or more selected from the group consisting of hierarchical cluster analysis (HCA), principal component analysis (PCA), and orthogonal partial least squares-discriminant analysis (OPLS-DA).

5. The method for identifying Calculus Bovis and a substitute thereof according to claim 1, wherein the significantly different CA component in step (2) is selected from the group consisting of CA, taurodeoxycholic acid (TDCA), taurolithocholic acid (TLCA), glycochenodeoxycholic acid (GCDCA), hyodeoxycholic acid (HDCA), and deoxycholic acid (DCA).

6. The method for identifying Calculus Bovis and a substitute thereof according to claim 1, wherein the discriminant function equation of the Calculus Bovis and the substitute thereof in step (3) comprises:
$$Y 1 = - 0.0 \; 34 S 1 + 0.345 S2 - 3.071 S 3 + 0.1 \; 21 S 4 + 0.5 \; 15 S 5 + 0.0 \; 71 S 6 - 91.575 ;$$
$$Y 2 = 0.2 \; 53 S 1 + 2.1 \; 42 S 2 + 4.9 \; 59 S 3 + 3.1 \; 61 S 4 - 0.446 S 5 - 0.207 S 6 - 401. 886 ;$$
$$Y 3 = 0.0 \; 15 S 1 + 0.0 \; 37 S 2 - 0.112 S 3 + 0.1 \; 45 S 4 - 0.023 S 5 + 0.0 \; 23 S 6 - 1.997 ;$$
$$Y 4 = 0.4 \; 72 S 1 - 0.78382 + 5.0 \; 61 S 3 + 2.3 \; 61 S 4 - 0.619 S 5 + 0.379 S 6 - 204. 836 ;$$
Y1 represents Calculus Bovis Artifactus, Y2 represents an ox bile powder, Y3 represents the Calculus Bovis, and Y4 represents In vitro cultivated Calculus Bovis; and
S1 represents CA, S2 represents TDCA, S3 represents TLCA, S4 represents GCDCA, S5represents HDCA, and S6 represents DCA.
